(19) United States
(12) Patent Application Publication
(10) Pub. No.: US 2024/0067883 A1
(43) Pub. Date: Feb. 29, 2024

(54) SYSTEM FOR PRODUCING HYDROCARBON COMPOUND AND METHOD FOR PRODUCING HYDROCARBON COMPOUND

(71) Applicants: MITSUBISHI HEAVY INDUSTRIES, LTD., Tokyo (JP); MITSUBISHI GAS CHEMICAL COMPANY, INC., Tokyo (JP)

(72) Inventors: Shinya MARUSHIMA, Tokyo (JP); Kenichi IRIE, Tokyo (JP); Takahiro KAMO, Tokyo (JP); Tadateru TANIOKA, Tokyo (JP); Yoshimasa ANDO, Tokyo (JP); Atsushi TSUTSUMI, Tokyo (JP)

(21) Appl. No.: 18/272,425

(22) PCT Filed: Feb. 24, 2022
(86) PCT No.: PCT/JP2022/007564

§ 371 Date: Jul. 14, 2023

(30) Foreign Application Priority Data
Feb. 26, 2021 (JP) 2021-029632

(51) Int. Cl. C10G 2/00

(52) U.S. Cl. CPC C10G 2/50

(57) ABSTRACT

Provided is a production system for producing a hydrocarbon compound, which is a hydrocarbon compound production system capable of managing an environmental load reducing effect derived from a raw material. The hydrocarbon compound production system includes a hydrogen production device that generates hydrogen, a carbon dioxide supply device that supplies a carbon dioxide, and a hydrocarbon compound production device that generates a hydrocarbon compound from each of the hydrogen generated by the hydrogen production device and the carbon dioxide supplied from the carbon dioxide supply device, wherein, on the basis of at least either one of respective environmental indicators of the hydrogen generated by the hydrogen production device and the carbon dioxide supplied from the carbon dioxide supply device, an environmental load level of the hydrocarbon compound generated by the hydrocarbon compound production device is categorized.

### TECHNICAL FIELD

The present invention pertains to a production system for producing a hydrocarbon compound such as methanol and to a production method therefor, and particularly relates to a hydrocarbon compound production system capable of managing an environmental load reducing effect derived from a raw material and to a hydrocarbon compound production method.

### BACKGROUND ART

While hydrogen is expected to be one of measures to reduce environmental loads, there is a case where a carbon dioxide (CO₂) is released in a process of generation thereof. For this reason, there are active discussions in an economic circle and an industrial circle about how to efficiently generate hydrogen with a small environmental load and what kind of hydrogen generation and use should be promoted. It is considered to focus attention on electric power serving as a power source in a generation process or the like and discriminate the generated hydrogen according to the environmental load.

For example, hydrogen generated by electrolysis of water using electric power derived from renewable energy is referred to as green hydrogen, hydrogen generated with a process of CO₂ capture and storage (CCS: carbon dioxide capture and storage) without releasing the carbon dioxide into an atmosphere can be regarded as not releasing CO₂ in terms of hydrogen generation and referred to as blue hydrogen, and hydrogen resulting from a generation process which uses fossil fuels as the raw material and in which CO₂ is released into the atmosphere is referred to as gray hydrogen.

Examples of a background art in the present technical field include a technology as in Patent Literature 1. In Patent Literature 1, “METHANOL PRODUCTION METHOD FOR EFFICIENTLY PRODUCING METHANOL FROM LOWER HYDROCARBONS SUCH AS NATURAL GAS” is disclosed.

### CITATION LIST

### Patent Literature

PTL 1: Japanese Unexamined Patent Application Publication No. 2012-219068

### SUMMARY OF INVENTION

### Technical Problem

As described above, in reducing environmental loads, it is important to give consideration to origins of raw materials and products and a production process.

However, an origin of hydrogen, which is the raw material to be used for, e.g., synthesis of recycled carbon methanol (ratio between fossil fuel-derived hydrogen and non-fossil fuel-derived hydrogen), changes over time, and consequently an origin of the produced methanol (ratio between fossil fuel-derived hydrogen and non-fossil fuel-derived hydrogen) becomes unclear, and an environmental load reducing effect of the produced methanol is uncertain.

Therefore, by recognizing the origin of the hydrogen used for the methanol production related to the environmental load, the environmental load reducing effect of the produced methanol becomes clear.

In Patent Literature 1 described above, no consideration is given to the origin of the hydrogen, and no mention has been made of categorization of the hydrogen based on an environmental indicator as described above.

It is therefore an object of the present invention to provide a production system for producing the hydrocarbon compound, which is the hydrocarbon compound production system capable of managing an environmental load reducing effect derived from the raw material, and a hydrocarbon compound production method using the same.

### Solution to Problem

To solve the problem described above, the present invention includes: a hydrogen production device that generates hydrogen; a carbon dioxide supply device that supplies the carbon dioxide; and a hydrocarbon compound production device that generates the hydrocarbon compound from each of the hydrogen generated by the hydrogen production device and the carbon dioxide supplied from the carbon dioxide supply device, wherein, on the basis of at least either one of respective environmental indicators of the hydrogen generated by the hydrogen production device and the carbon dioxide supplied from the carbon dioxide supply device, an environmental load level of the hydrocarbon compound generated by the hydrocarbon compound production device is categorized.

The present invention is also a hydrocarbon compound production method for generating the hydrocarbon compound using each of hydrogen and the carbon dioxide as the raw material, and the hydrocarbon compound production method includes: categorizing an environmental load level of the generated hydrocarbon compound on the basis of at least either one of respective environmental indicators of the hydrogen and the carbon dioxide each used as the raw material.

### Advantageous Effects of Invention

According to the present invention, it is possible to implement a production system for producing the hydrocarbon compound, which is the hydrocarbon compound production system capable of managing an environmental load reducing effect derived from a raw material, and a hydrocarbon compound production method using the same.

This allows the produced hydrocarbon compound to be categorized by environmental load levels.

Problems, configurations, and effects other than those described above will be clarified by the following description of embodiments.

### BRIEF DESCRIPTION OF DRAWINGS

FIG. 1 is a diagram illustrating a schematic configuration of a hydrocarbon compound production system according to a first embodiment;

FIG. 2 is a diagram illustrating a schematic configuration of the hydrocarbon compound production system according to a second embodiment;

FIG. 3 is a diagram illustrating a schematic configuration of the hydrocarbon compound production system according to a third embodiment;

FIG. 4 is a diagram illustrating a schematic configuration of the hydrocarbon compound production system according to a fourth embodiment;

FIG. 5 is a diagram illustrating a schematic configuration of the hydrocarbon compound production system according to a fifth embodiment;

FIG. 6 is a diagram illustrating an environmental indicator table according to the fifth embodiment.

### DESCRIPTION OF EMBODIMENTS

Using the drawings, embodiments of the present invention will be described below. In each of the drawings, the same components are denoted by the same reference signs, and a detailed description of overlapping portions is omitted.

### First Embodiment

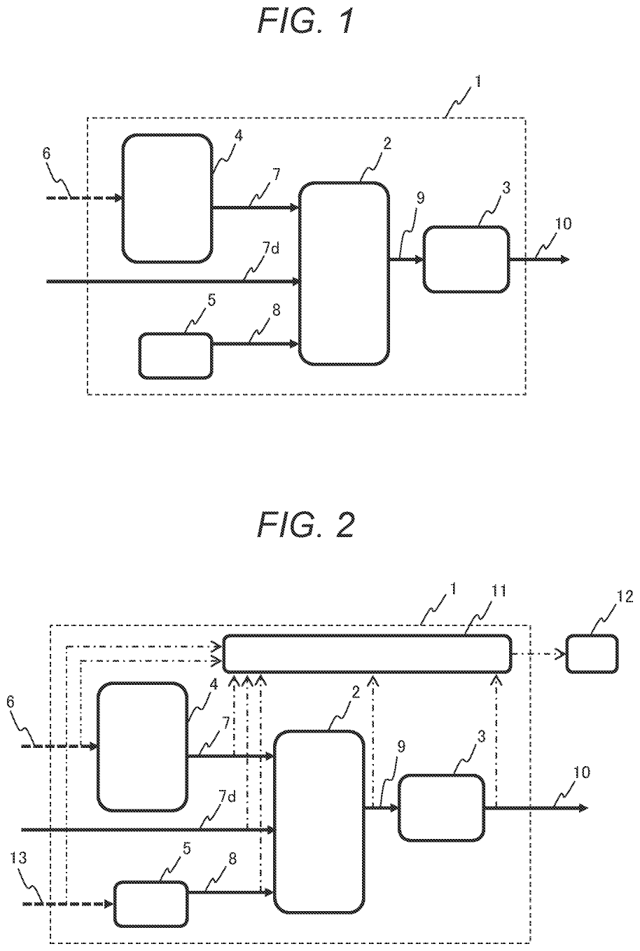

Referring to FIG. 1, a description will be given of the hydrocarbon compound production system and a hydrocarbon compound production method using the same according to a first embodiment of the present invention. FIG. 1 is a diagram illustrating a schematic configuration of the hydrocarbon compound production system in the present embodiment.

In the present embodiment, a basic concept of the present invention will be described.

As described above, the origin of hydrogen to be used for the synthesis of the recycled carbon methanol (ratio between fossil fuel-derived hydrogen and non-fossil fuel-derived hydrogen) changes over time, and consequently the origin of the produced methanol (ratio between the fossil fuel-derived hydrogen and the non-fossil fuel-derived hydrogen) may become unclear.

Examples of the origin of hydrogen include hydrogen produced only from renewable energy electric power (including electric power regarded as the renewable electric power), hydrogen produced using system electric power, stored hydrogen, by-product hydrogen or hydrogen with CCS, hydrogen combined with the former, and the like.

To clarify the origin of the produced hydrogen, the following can be considered: (1) a temporal change of the origin of the supplied hydrogen is measured, and the origin of the produced methanol is calculated; (2) the origin of the methanol in a methanol storage tank is calculated; and (3) the methanol can be traded according to the origin, and a premium or carbon tax according to the origin can be added.

Accordingly, as illustrated in FIG. 1, a methanol production system 1 in the present embodiment includes, as main components, a hydrogen production device 4 that generates hydrogen 7, a carbon dioxide supply device 5 that supplies a carbon dioxide 8, and a methanol production device 2 that generates methanol 9 from each of the hydrogen 7 generated by the hydrogen production device 4 and the carbon dioxide 8 supplied from the carbon dioxide supply device 5.

The hydrogen production device 4 uses electric power supplied from an electric power source 6 to generate the hydrogen 7.

To the methanol production device 2, in addition to a system that supplies the hydrogen 7 generated by the hydrogen production device 4, a system that supplies hydrogen 7d generated with a CO₂ capture and storage process (CCS) is also connected, and it is also possible to generate the methanol 9 using the hydrogen 7d generated with the CO₂ capture and storage process (CCS) instead of using the hydrogen 7 generated by the hydrogen production device 4.

The methanol 9 generated by the methanol production device 2 is temporarily stored in a storage device 3.

The methanol production system 1 in the present embodiment categorizes herein an environmental load level of the methanol 9 generated by the methanol production device 2 on the basis of at least either one of environmental indicators of the hydrogen 7 generated by the hydrogen production device 4 and the carbon dioxide 8 supplied from the carbon dioxide supply device 5. When the hydrogen 7d generated with the CO₂ capture and storage process (CCS) is used, the environmental load level of the methanol 9 is categorized on the basis of the environmental indicator of the hydrogen 7d.

By categorizing the environmental load level of the methanol 9 corresponding to a product on the basis of the respective environmental indicators of the hydrogen 7 or 7d and the carbon dioxide 8 each corresponding to the raw material, it is possible to categorize methanol 10 supplied (shipped) from the storage device 3 by the environmental load levels.

### Second Embodiment

Referring to FIG. 2, a description will be given of the hydrocarbon compound production system and the hydrocarbon compound production method using the same according to the second embodiment of the present invention. FIG. 2 is the diagram illustrating the schematic configuration of the hydrocarbon compound production system in the present embodiment.

In the present embodiment, a system configuration example including a control device 11 in addition to the configuration in the first embodiment (FIG. 1) will be described.

The methanol production system 1 in the present embodiment includes the control device 11 as illustrated in FIG. 2, and the control device 11 monitors each of information on the electric power source 6 of the hydrogen production device 4, information on a carbon dioxide supply source 13 of the carbon dioxide supply device 5, information on the hydrogen 7 generated by the hydrogen production device 4, information on the hydrogen 7d generated with the CO₂ capture and storage process (CCS), information on the carbon dioxide 8 supplied from the carbon dioxide supply device 5, information on the methanol 9 generated in the methanol production device 2, and information on the methanol 10 supplied (shipped) from the storage device 3 to the outside. Each item of the information is the environmental indicator considering the origins of the raw materials and a product as well as a production process.

The control device 11 categorizes the methanol 10 supplied (shipped) from the storage device 3 to the outside by the environmental load levels on the basis of each item of the monitored information (environmental indicator), and outputs (displays) a result thereof to (on) an output device 12 to notify a manager. Note that the output device 12 can also be embedded in the methanol production system 1.

### Third Embodiment

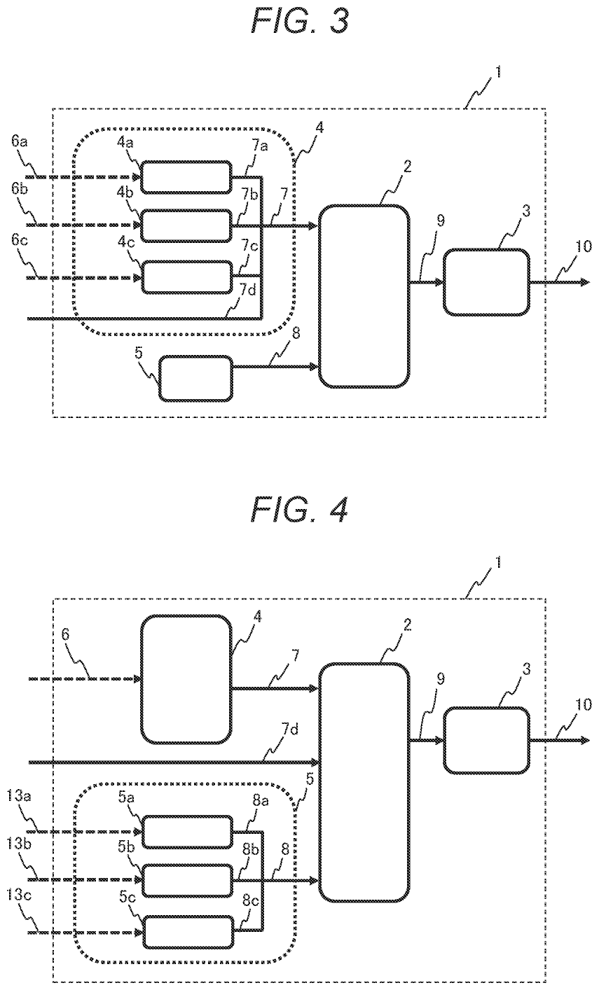

Referring to FIG. 3, a description will be given of the hydrocarbon compound production system and the hydrocarbon compound production method using the same according to the third embodiment of the present invention. FIG. 3 is the diagram illustrating the schematic configuration of the hydrocarbon compound production system in the present embodiment.

In the present embodiment, the system configuration example that generates, in the configuration in the first embodiment (FIG. 1), the hydrogen 7 using electric power from a plurality of electric power sources will be described.

As illustrated in FIG. 3, in the methanol production system 1 in the present embodiment, the hydrogen production device 4 is configured to include a hydrogen production device 4a that uses electric power supplied from an electric power source 6a to generate hydrogen 7a, a hydrogen production device 4b that uses electric power supplied from an electric power source 6b to generate hydrogen 7b, and a hydrogen production device 4c that uses electric power supplied from an electric power source 6c to generate hydrogen 7c.

In addition, in the same manner as in the first embodiment (FIG. 1), a system that supplies hydrogen 7d generated with the CO₂ capture and storage process (CCS) is also connected.

The electric power source 6a is that of the electric power derived from the renewable energy, and the hydrogen 7a generated in the hydrogen production device 4a is green hydrogen. Note that the renewable energy also includes electric power regarded as the renewable energy, i.e., so-called deemed renewable energy electric power.

The electric power source 6b is that of electric power derived from the system electric power, and the hydrogen 7b generated in the hydrogen production device 4b is a hydrogen mixture of the green hydrogen, blue hydrogen, and gray hydrogen.

The electric power source 6c is that of electric power generated using the fossil fuels as the raw material, and the hydrogen 7c generated in the hydrogen production device 4c is the gray hydrogen. In an electric power generation process in the electric power source 6c, the carbon dioxide is released into the atmosphere.

The hydrogen 7d generated with the CO₂ capture and storage process (CCS) is the blue hydrogen.

The methanol production system 1 in the present embodiment is thus configured, and can determine the environmental indicator of the hydrogen 7 generated by the hydrogen production device 4 on the basis of information on the electric power sources 6a to 6c of the hydrogen production devices 4a to 4c.

### Fourth Embodiment

Referring to FIG. 4, a description will be given of the hydrocarbon compound production system and the hydrocarbon compound production method using the same according to the fourth embodiment of the present invention. FIG. 4 is the diagram illustrating the schematic configuration of the hydrocarbon compound production system in the present embodiment.

In the present embodiment, the system configuration example that uses, in the configuration in the first embodiment (FIG. 1), a plurality of carbon dioxide supply devices to supply the carbon dioxide 8 will be described.

In the methanol production system 1 in the present embodiment, as illustrated in FIG. 4, the carbon dioxide supply device 5 is configured to include a carbon dioxide supply device 5a that temporarily stores the carbon dioxide supplied from a carbon dioxide supply source 13a to supply a carbon dioxide 8a, a carbon dioxide supply device 5b that temporarily stores the carbon dioxide supplied from a carbon dioxide supply source 13b to supply a carbon dioxide 8b, and a carbon dioxide supply device 5c that temporarily stores a carbon dioxide supplied from a carbon dioxide supply source 13c to supply a carbon dioxide 8c.

The carbon dioxide supply source 13a is that of atmosphere-derived CO₂ collected from the atmosphere.

The carbon dioxide supply source 13b is that of biomass-fuel-derived CO₂ generated through combustion of a biomass fuel.

The carbon dioxide supply source 13c is that of fossil fuel-derived CO₂ generated through combustion of the fossil fuels.

The methanol production system 1 in the present embodiment is thus configured, and can determine an environmental indicator of the carbon dioxide 8 supplied from the carbon dioxide supply device 5 on the basis of the information on the carbon dioxide supply sources 13a to 13c of the carbon dioxide supply devices 5a to 5c.

### Fifth Embodiment

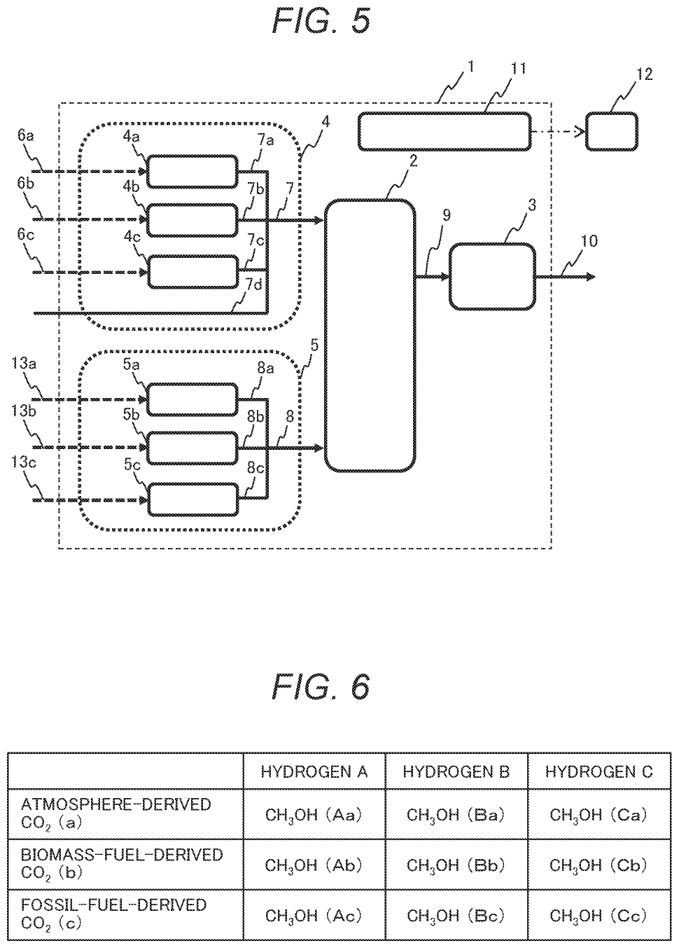

Referring to FIGS. 5 and 6, a description will be given of the hydrocarbon compound production system and the hydrocarbon compound production method using the same according to the fifth embodiment of the present invention. FIG. 5 is the diagram illustrating the schematic configuration of the hydrocarbon compound production system in the present embodiment. FIG. 6 is the diagram illustrating the environmental indicator table in the present embodiment.

In the present embodiment, the system configuration example including all of the individual configurations described in the first embodiment (FIG. 1) to the fourth embodiment (FIG. 4) will be described.

As illustrated in FIG. 5, the methanol production system 1 in the present embodiment is configured to include all of the individual configurations described in the first embodiment (FIG. 1) to the fourth embodiment (FIG. 4). The individual configurations and functions thereof are exactly as described in the individual embodiments, and a repeated description thereof is omitted.

Note that, in FIG. 5, for easier understanding of the configuration, a dash-dot line indicating information monitored by the control device 11 as in FIG. 2 is omitted.

### Definitions/Effects of Environmental Indicators

Using FIG. 6, a description will be given of an environmental indicator table in the present embodiment. In FIG. 6, hydrogen A indicates the green hydrogen, hydrogen B indicates the blue hydrogen, and hydrogen C indicates the gray hydrogen.

As illustrated in FIG. 6, in the present embodiment, on the basis of the respective environmental indicators of both of the hydrogen 7 and the carbon dioxide 8 each serving as the raw material, the environmental load level of the produced methanol 9 is categorized.

For example, the methanol 9 generated from “HYDROGEN A (green hydrogen)” and “ATMOSPHERE-DERIVED CO₂ (a)” is categorized to “CH₃OH (Aa)”. Likewise, the methanol 9 is categorized to any of methanols of nine different origins according to the combinations of the “HYDROGEN A (green hydrogen)”, “HYDROGEN B (blue hydrogen)”, and “HYDROGEN C (gray hydrogen)”, which are three categories of the hydrogen 7, and the “ATMOSPHERE-DERIVED CO₂ (a)”, “BIOMASS-FUEL-DERIVED CO₂ (b)”, and “FOSSIL FUEL-DERIVED CO₂ (c)”, which are three categories of the carbon dioxide 8.

By thus using the origins of the hydrogen and the carbon dioxide each serving as the raw material to categorize the environmental load level of the generated methanol, an environmental value of the methanol is clarified.

Note that the origins of the hydrogen 7 and the carbon dioxide 8 each supplied to the methanol production device 2 may change with time.

In such a case, it is also possible that a temporal change of the environmental indicator of the hydrogen 7 and a temporal change of the environmental indicator of the carbon dioxide 8 are monitored using the control device 11 and, on the basis of the monitored temporal changes of the respective environmental indicators of the hydrogen 7 and the carbon dioxide 8, the component ratio of the methanol 9 in the storage device 3 is determined. Thus, it is possible to recognize the origin of the methanol to be distributed. It is conceivable to place at least two or more of the storage device 3 described in the foregoing embodiment. For example, it is also conceivable to place nine of the storage device 3 in the combinations illustrated in FIG. 6 and allow separate storage to be performed according to the origins of the raw material hydrogen and CO₂. In the future, when environmental value standards such as a green hydrogen content are determined, it is also conceivable to perform separate storage in the storage device for the methanol satisfying the environmental standards and in the storage device for the methanol other than that.

It should be noted that the present invention is not limited to the examples described above, and includes various modification examples. For example, the examples described above have been described in detail to simply describe the present invention, and are not necessarily required to include all the described configurations. In addition, part of the configuration of one example can be replaced with the configurations of other examples, and in addition, the configuration of the one example can also be added with the configurations of other examples. In addition, part of the configuration of each of the examples can be subjected to addition, deletion, and replacement with respect to other configurations.

### REFERENCE SIGNS LIST

1: methanol production system (hydrocarbon compound production system), 2: methanol production device (hydrocarbon compound production device), 3: storage device, 4, 4a, 4b, 4c: hydrogen production device, 5, 5a, 5b, 5c: carbon dioxide supply device, 6, 6a, 6b, 6c: electric power source, 7, 7a, 7b, 7c: hydrogen, 8, 8a, 8b, 8c: carbon dioxide, 9, 10: methanol, 11: control device, 12: output device, 13, 13a, 13b, 13c: carbon dioxide supply source

1. A hydrocarbon compound production system comprising:
a hydrogen production device that generates hydrogen;
a carbon dioxide supply device that supplies a carbon dioxide; and
a hydrocarbon compound production device that generates a hydrocarbon compound from each of the hydrogen generated by the hydrogen production device and the carbon dioxide supplied from the carbon dioxide supply device, wherein,
on the basis of at least either one of respective environmental indicators of the hydrogen generated by the hydrogen production device and the carbon dioxide supplied from the carbon dioxide supply device, an environmental load level of the hydrocarbon compound generated by the hydrocarbon compound production device is categorized.

2. The hydrocarbon compound production system according to claim 1, wherein the environmental indicator of the hydrogen generated by the hydrogen production device is categorized into one of three categories of hydrogen generated by electrolysis of water using electric power derived from renewable energy, hydrogen generated with a carbon dioxide capture and storage process, and hydrogen resulting from a generation process which uses fossil fuels as a raw material and in which the carbon dioxide is released into an atmosphere.

3. The hydrocarbon compound production system according to claim 1, wherein, on the basis of information on an electric power source of the hydrogen production device, the environmental indicator of the hydrogen generated by the hydrogen production device is determined.

4. The hydrocarbon compound production system according to claim 2, further comprising:
a storage device that stores the hydrocarbon compound generated by the hydrocarbon compound production device; and
a control device that monitors a temporal change of the environmental indicator of the hydrogen generated by the hydrogen production device, wherein
the control device determines a component ratio of the hydrocarbon compound in the storage device on the basis of the monitored temporal change of the environmental indicator of the hydrogen.

5. The hydrocarbon compound production system according to claim 1, wherein, on the basis of information on a carbon dioxide supply source of the carbon dioxide supply device, the environmental indicator of the carbon dioxide supplied from the carbon dioxide supply device is determined.

6. The hydrocarbon compound production system according to claim 5, wherein the environmental indicator of the carbon dioxide supplied from the carbon dioxide supply device is categorized into one of three categories of a carbon dioxide collected from an atmosphere, a carbon dioxide generated through combustion of a biomass fuel, and a carbon dioxide generated through combustion of the fossil fuels.

7. The hydrocarbon compound production system according to claim 5, further comprising:
the storage device that stores the hydrocarbon compound generated by the hydrocarbon compound production device; and
a control device that monitors a temporal change of the environmental indicator of the carbon dioxide supplied from the carbon dioxide supply device, wherein
the control device determines a component ratio of the hydrocarbon compound in the storage device on the basis of the monitored temporal change of the environmental indicator of the carbon dioxide.

8. A hydrocarbon compound production method for generating a hydrocarbon compound using each of hydrogen and a carbon dioxide as the raw material, the hydrocarbon compound production method comprising:
categorizing an environmental load level of the generated hydrocarbon compound on the basis of at least either one of respective environmental indicators of the hydrogen and the carbon dioxide each used as the raw material.

9. The hydrocarbon compound production method according to claim 8, wherein the environmental indicator of the hydrogen used as the raw material is categorized into one of three categories of hydrogen generated by electrolysis of water using electric power derived from renewable energy, hydrogen generated with a carbon dioxide capture and storage process, and hydrogen resulting from a generation process which uses the fossil fuels as the raw material and in which the carbon dioxide is released into an atmosphere.

10. The hydrocarbon compound production method according to claim 8, wherein, on the basis of information on an electric power source used in a production process of the hydrogen used as the raw material, the environmental indicator of the hydrogen used as the raw material is determined.

11. The hydrocarbon compound production method according to claim 9, wherein
a temporal change of the environmental indicator of the hydrogen used as the raw material is monitored, and,
on the basis of the monitored temporal change of the environmental indicator of the hydrogen, a component ratio in the storage device that stores the generated hydrocarbon compound is determined.

12. The hydrocarbon compound production method according to claim 8, wherein, on the basis of information on a supply source of the carbon dioxide used as the raw material, the environmental indicator of the carbon dioxide used as the raw material is determined.

13. The hydrocarbon compound production method according to claim 12, wherein the environmental indicator of the carbon dioxide used as the raw material is categorized into one of three categories of a carbon dioxide collected from an atmosphere, a carbon dioxide generated through combustion of the biomass fuel, and a carbon dioxide generated through combustion of the fossil fuels.

14. The hydrocarbon compound production method according to claim 12, wherein
a temporal change of the environmental indicator of the carbon dioxide used as the raw material is monitored, and,
on the basis of the monitored temporal change of the environmental indicator of the carbon dioxide, a component ratio in the storage device that stores the generated hydrocarbon compound is determined.
